(19) United States
(12) Patent Application Publication
(10) Pub. No.: US 2007/0160975 A1
(43) Pub. Date: Jul. 12, 2007

(54) Method for the Identification of Drugs to Treat Stroke at Delayed Timepoints

(75) Inventors: Javier Cuevas, Lutz, FL (US); Keith R. Pennypacker, Wesley Chapel, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/621,196

(22) Filed: Jan. 9, 2007

Related U.S. Application Data
(60) Provisional application No. 60/766,301, filed on Jan. 9, 2006.

(51) Int. Cl. C12Q 1/00; G01N 33/567

(52) U.S. Cl. 435/4; 435/7.2

(57) ABSTRACT

A method of in vitro screening for compounds for treating strokes at delayed timepoints of administration following the onset of stroke. In a first aspect the method includes the step of contacting neurons with azide/deoxyglucose to induce ischemia, contacting with a compound of interest at a later timepoint and assessing neuronal death. A reduction in neuronal death at the later timepoint relative to one or more controls indicates the compound of interest is a candidate for stroke treatment in vivo at delayed timepoints. In another aspect the method includes the step of contacting neurons with an inflammatory agent, such as a lipopolysaccharide, contacting with a compound of interest and assessing the inflammatory response. The methods allow for screening of compounds at higher throughput and lower cost than in vivo methods currently used. Compounds exhibiting promise in the in vitro system can be further characterized by traditional in vivo screening.

### CROSS REFERENCE TO RELATED APPLICATIONS

This application claims priority to currently pending U.S. Provisional Patent Application 60/766,301, entitled, “Identification of Drugs for Efficacy to Treat Stroke at Delayed Timepoints”, filed Jan. 09, 2006, the contents of which are herein incorporated by reference.

### STATEMENT OF GOVERNMENT INTEREST

This invention was made with Government support under Grant No. NIH NS39141 awarded by the National Institutes of Health. The Government has certain rights in the invention.

### FIELD OF INVENTION

This invention relates to the treatment of stroke. More specifically, this invention relates methods to identify drugs for the treatment of stroke where the drugs are to be administered at delayed timepoints relative to the incidence of the stroke.

### BACKGROUND OF THE INVENTION

Stroke is the leading cause of severe disability and the third leading cause of death in the United States of America (AHA, 2005). Intravenous application of recombinant tissue plasminogen activator (tPA), a thrombolytic agent, is the only FDA approved treatment for stroke and has a very limited therapeutic time window (National Institute of Neurological Disorders and Stroke (NINDS): (1995) N Engl J Med 333 (24): 1581-7). This “clotbuster” must be administered within three hours of stroke onset (Albers, G W, Amarenco, P, Easton, J D, Sacco, R L. and Teal, P. (2004) Chest 126(3 Suppl): 483S-512S), and can produce possible adverse effects such as hemorrhage and reperfusion damage from oxygen free radicals (Hacke, W, et al. (1999) Neurology 53(7 Suppl 4): S3-14; Kumura, E, et al. (1996) Am J Physiol 270(3 Pt 1): C748-52; Peters, O, et al. (1998) J Cereb Blood Flow Metab 18(2): 196-205). The limitations and adverse effects of tPA have stimulated the search for alternative treatments for stroke.

When a cerebral embolic stroke occurs, a thrombus blocks blood perfusion to the brain and triggers a series of events that ultimately result in neuronal death. The disruption in blood supply directly results in the cessation of oxygen and nutrient delivery, which metabolically compromises the neurons and produces an infarction. The infarct zone contains two regions associated with ischemic cell death. The center of the infarction or “core” is the area directly affected by the decrease in blood perfusion, and is where the greatest concentration of cell death can be found. Surrounding the core is the penumbra, a region with diminished blood flow but where collaterals provide some oxygen and nutrients. However, perfusion in the penumbra is sufficiently reduced that physiological function is arrested and some degeneration of neurons occurs (Ginsberg, MD. (2003) Stroke 34(1): 2 14-23).

Neuronal death is enhanced by secondary inflammation caused by the immune response in the penumbra. The inflammatory response is primarily from resident activated microglia and infiltrating macrophages, which enter the central nervous system through the degrading blood brain barrier (Stoll, G, et al. (1998) Prog Neurobiol 56 (2): 149-71). Reactive astrocytes and microglia exacerbate cerebral inflammation via their production of pro-inflammatory cytokines and chemokines (Trendelenburg, G, et al. (2005) Glia 50 (4): 307-20). These immune cells, which normally protect the brain via destruction of pathogens and promotion of tissue repair, become overactivated, and further promote the expansion of tissue damage by releasing high levels of nitric oxide (NO), glutamate, tumor necrosis factor-alpha (TNF-α), and interleukin-1 IL-1) (Bal-Price, A, et al. (2001) J Neurosci 21(17): 6480-91; Heales, S J, et al. (1999) Biochim Biophys Acta 1410 (2): 215-28; Hertz, L, et al. (2001) Neurochem mt 39 (3): 227-52).

At the present time, tPA is the only FDA-approved treatment for stroke. tPA must be administered within 3 hours of the stroke onset. This very limited therapeutic time window is a significant limitation in the use of tPA. Additional drugs to treat for stroke where the drugs offer an extended treatment window would significantly enhance treatment options. Screening for such drugs currently requires the use of in vivo models that can be both expensive and time consuming. it would be highly desirable to have an in vitro model of stroke to allow for the screening of the efficacy of drugs for the treatment of stroke where the drugs are to be administered at a delayed timepoint after the onset of stroke. The present invention meets this important need and others as will become apparent.

### SUMMARY OF INVENTION

A method of in vitro screening for compounds for treating strokes at delayed timepoints of administration following the onset of stroke. In a first aspect the method includes the step of contacting neurons with azide/deoxyglucose to induce ischemia. The neurons are then contacted with a compound of interest at a later timepoint to simulate the effect of treating for stroke. Following contact with the compound of interest, neuronal death is assessed to evaluate the effect of the compound of interest on the neurons induced for ischemia. A reduction in neuronal death at the later timepoint relative to one or more controls indicates the compound of interest is a candidate for stroke treatment in vivo at delayed timepoints. In another aspect the method includes the step of contacting neurons with an inflammatory agent such as a lipopolysaccharide to induce an inflammatory response in the neurons. The neurons are then contacted with a compound of interest at a later timepoint to simulate the effect of treating for stroke with that compound. Following contact with the compound of interest, reduction in the inflammatory response are assessed. Reductions in the inflammatory response can be measured by assessing TNF-α of nitric oxide levels. A reduction in the inflammatory response in the contacted neurons at the later timepoint relative to one or more controls indicates the compound of interest is a candidate for stroke treatment in vivo at delayed timepoints. The methods taught herein allow for the screening of compounds at higher throughput and lower cost than can be achieved by in vivo methods currently used. Compounds exhibiting promise in the in vitro system can be further characterized by traditional in vivo screening.

In a first aspect the present invention provides a method of in vitro screening of compounds for the treatment of stroke including the steps of contacting neurons with azide/deoxyglucose to induce ischemia, contacting the neurons with a compound of interest at about 6 hours after the contact of the neurons with azide/deoxyglucose and measuring neuronal death at about 24 hours after the induction of ischemia. A reduction in neuronal death indicates that a compound of interest is a treatment candidate for the treatment of stroke. In certain embodiments the compound of interest is a sigma receptor agonist.

In a second aspect the present invention provides a method of in vitro screening of compounds for the treatment of stroke including the steps of inducing ischemia in a population of in vitro neurons, contacting the neurons with a compound of interest after the induction of ischemia and measuring neuronal death at about 6 or more hours after the induction of ischemia. A reduction in neuronal death indicates that a compound of interest is a treatment candidate for the treatment of stroke. In certain embodiments ischemia is induced with azide/deoxyglucose. The compound of interest can be a sigma receptor agonist. In certain embodiments the neurons are contacted with the compound of interest at about 1, 2, 3, 4, 6, 8, 10, 12, 18, 24, 36 or 48 or more hours after the induction of ischemia. In certain embodiments neuronal death is measured at about 2, 3, 4, 6, 8, 10, 12, 18, 24, 36, 48, 60, 72, 84, or 96 or more hours after the induction of ischemia.

In a third aspect the present invention provides a method of in vitro screening of compounds for the treatment of stroke including the steps of contacting neurons with lipopolysaccharide to induce an inflammatory response, contacting the neurons with a compound of interest at about 6 hours after the contact of the neurons with lipopolysaccharide and measuring the inflammatory response in the cells at about 24 hours after the induction of the inflammatory response, whereby a reduction in the inflammatory response indicates that a compound of interest is a treatment candidate for the treatment of stroke. In certain embodiments the step of measuring the inflammatory response comprises measuring TNF-α levels. A reduction in TNF-α levels indicates a reduced inflammatory response. In alternative embodiments step of measuring the inflammatory response comprises measuring nitric oxide levels. A reduction in nitric oxide levels indicates a reduced inflammatory response.

In a fourth aspect the present invention provides a method of in vitro screening of compounds for the treatment of stroke including the steps of inducing an inflammatory response in an in vitro population of neurons, contacting the neurons with a compound of interest after the induction of the inflammatory response and measuring the inflammatory response in the cells at about 6 or more hours after the induction of the inflammatory response. A reduction in the inflammatory response indicates that a compound of interest is a treatment candidate for the treatment of stroke. In certain embodiments the step of measuring the inflammatory response comprises measuring TNF-α levels. A reduction in TNF-α levels indicates a reduced inflammatory response. In alternative embodiments step of measuring the inflammatory response comprises measuring nitric oxide levels. A reduction in nitric oxide levels indicates a reduced inflammatory response. In certain embodiments the neurons are contacted with the compound of interest at about 1, 2, 3, 4, 6, 8, 10, 12, 18, 24, 36 or 48 or more hours after the induction of the inflammatory response. In certain embodiments the inflammatory response in the neurons is measured at about 2, 3, 4, 6, 8, 10, 12, 18, 24, 36, 48, 60, 72, 84, or 96 or more hours after the induction of the inflammatory response.

### BRIEF DESCRIPTION OF THE DRAWINGS

For a fuller understanding of the invention, reference should be made to the following detailed description, taken in connection with the accompanying drawings, in which:

FIG. 1 is a graph illustrating that sigma ligands are neuroportective when appliced during or following ischemia. Bar graph (mean±SEM) of relative LDH concentration detected for the conditions indicated 24 hours after the ischemia. DTG was applied during the ischemia (Acute DTG) or following the ischemia (Delayed DTG). Levels of LDH were normalized to those measured in dishes of neurons not exposed to either ischemia or DTG during the 30 hour period. For all samples, n=5.

FIG. 2 is a graph illustrating that sigma receptor activation reduces LPS-induced cytotoxin production. DTG dose dependently reduced LPS stimulated TNF-α and nitric oxide release from rate primary microglial cultures. Nitric oxide levels were quantified using the Griess reaction while TNF-α levels were measured by ELISA.

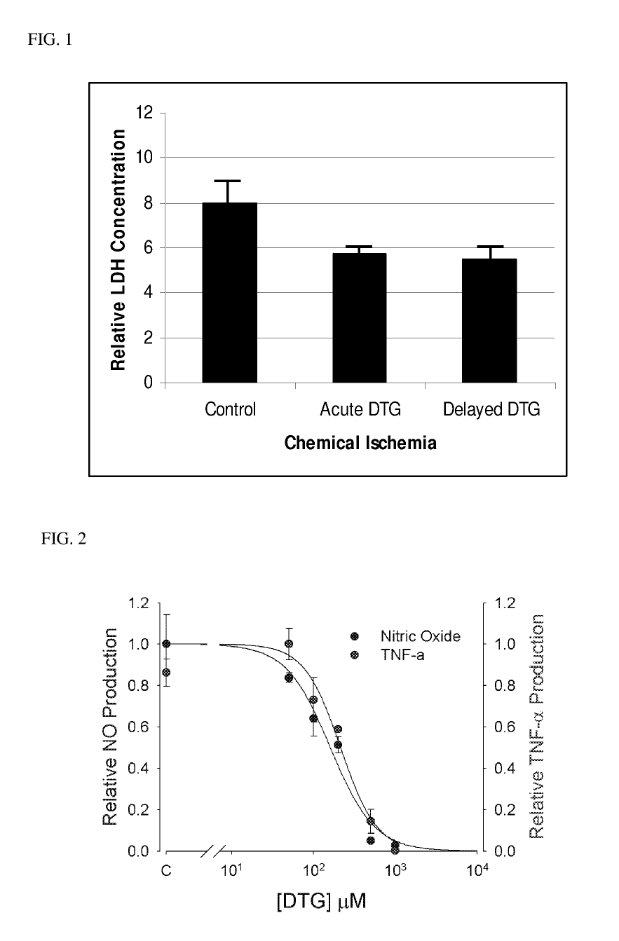

### DETAILED DESCRIPTION OF THE PREFERRED EMBODIMENT

Stroke is a third leading killer in the United States and first in cause of disability. The only drug available for treatment of stroke is tissue plasiminogen activator, which is a clot degrading agent. There is a need to discover new agents to reduce the injury to the brain to increase not only survival but also to decrease the severity of the disability. Fresh approaches are needed in order to discover novel agents to thwart this major pathology.

Since most people are unaware that they have incurred stroke for hours to days afterward, our laboratory has focused on treating stroke at delayed timepoints in a rat model. In stroke injury, neurons degenerate directly from lack of oxygen and excitotoxicity. Additionally, an inflammatory response to the degenerating neurons causes further damage to the brain. It has been shown in vivo rat models that stroke may be successfully treated at 24-48 hours after the stroke reducing the area of brain damage by 80%. The effective treatment regimen must enhance neurosurvival and inhibit the inflammatory response to the damaged brain tissue. The next problem arises in how to discover drugs with these two actions. Screening drugs in vivo models of stroke are costly and time-consuming. We have found that using primary rat neuronal and microglial cultures to test compounds for their neurosurvival and anti-inflammatory actions is a viable approach. We have found that compounds that enhance neurosurvival in vivo in stroke at delayed timepoints are also able to protect neurons against chemical ischemia, a toxicant used as an in vitro model of stroke. Azide/deoxyglucose inhibits mitochondrial metabolism and simulates a stroke-like environment for cultured neurons. Primary neurons would be treated with the compound of interest 6 hours after exposure to azide/deoxyglucose. Neuronal death would be measured 24 hours after chemical ischemia.

To gauge anti-inflammatory action, primary microglial cultures will be treated with lipopolysaccharide, which elicits a robust inflammatory response from these cells. Compounds that are effective agonist stroke at delayed timepoints are able to block the LPS-induced inflammatory response in microglial cells. The inflammatory cytokine, TNF-α and nitric oxide are two inflammatory agents to be measured to determine anti-inflammatory action of the compound. We have found that agents used to treat stroke at delayed timepoints block the production and release of these cytotoxins. Using these two in vitro screening methods, other compounds that will be useful in treating stroke will be discovered.

The invention is described below in examples which are intended to further describe the invention without limitation to its scope.

### EXAMPLE 1

Screening Methods

TNF-α ELISA-Capture antibody (#MAB510 anti-rat antibody TNF R&D biosysterns) will be diluted with 2 μg/ml in coating buffer and incubated at 4° C. overnight. The capture antibody will then coat a high protein binding 96 well plate. Each well of the 96 well plate will be aspirated and washed three times with wash buffer. The 96 well plate will be blocked by adding 300 μl PBS containing 1% BSA, 5% sucrose, and 0 05% sodium azide to each well. The 96 well plate will then be incubated for a minimum of 1 hour and then the blocking buffer will be aspirated by washing the wells once with wash buffer. A 2048 pg/ml stock of recombinant rat TNF (obtained from Preprotech) will be prepared by aliquoting recombinant rat TNF at 20 ng/tube and then adding to 976 μl of DMEM. A standard curve will be prepared by serially diluting the 2048 pg/ml stock In polyproylene microfuge tubes using DMEM as the diluent. 100 μl of the standards as well as the unknowns will be added to the previously coated wells and then the 96 well plate will be incubated for 1 hour at room temperature. After 1 hr of incubation the wells will be aspirated and then washed three times with wash buffer. Biotinylated detection antibody (#BAF5IO biotinylated anti-rat TNF antibody R&D biosystems) will be diluted with dilution buffer to 100 ng/ml and 100 μl will be added to each well. The wells will then be covered with parafilm and incubated for 2 hours at room temperature. After incubation the wells will be washed three times with wash buffer. Streptavidin-HRP will be diluted 1/25,000 in dilution buffer and 100 μl will be added to each well followed by 20 minutes incubation at room temperature. Following incubation the wells will be washed three times with wash buffer. 100 μ3,3′,5,5′-tetramethyl-benzidine (TMB) liquid will be added and incubated for 5 minutes. During incubation, direct light will be avoided 50 μl of 1 M H₂SO₄ will be added to each well and optical density will be determined within 30 minutes using a microplate reader set at 450 nm.

LDH Assay procedure: 100 μl of each test media will be pipetted into successive wells of a 96 well plate, ensuring proper labeling for identification of each solution The LDH assay reagent will be prepared just prior to the reaction and will be made by mixing 11.25 ml of reconstituted catalyst solution with 250 μl of INT dye solution in a tube covered with foil. The reaction, upon addition of reagent, is light sensitive and thus the reagent will only be added to each test media in darkness After the addition of LDH reagent the well plate will be covered in foil. The reaction will be allotted 30 minutes and the foil cover will only be removed just prior to placing the 96 well plate into a spectrophotometer for analysis.

Nitric Oxide Determination: The supematant from treated primary rat rnicrogiia cultures will be harvested, placed in 1.5 ml micro-centrifuge tubes and then centrifuged in a bench top micro-centrifuge at 13,000×g for 1 minute. The supernatant will be withdrawn and placed in a clean, labeled micro-centrifuge tube. Standards (between 100 μM and 1 μM) will be prepared by diluting stock 1 mM Sodium Nitrite, contained in the kit, with DMEM in 1.5 ml micro centrifuge tubes. 100 μl of cell free supernatant and 100 μl of standards will be added to an optically clean 96 well micro titer plate. Griess Reagent will be prepared by mixing equal volumes of 1% Sulfanilic Acid (in 5% Phosphoric acid) and 0.1% N-(1-naphthyl) ethylene-diamine, dihydrochlorlde and then diluted with 7.5 μl water per 1 μl Griess Reagent. 100 μl diluted Griess reagent will be added to each well followed by 30 minutes of incubation at room temperature. The absorbance at 458 nm will be measured in a spectrophotometer capable of reading 96 well plates.

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of in vitro screening of compounds for the treatment of stroke comprising the steps of:
contacting neurons with azide/deoxyglucose to induce ischemia;
contacting the neurons with a compound of interest at about 6 hours after the contact of the neurons with azide/deoxyglucose; and
measuring neuronal death at about 24 hours after the induction of ischemia, whereby a reduction in neuronal death indicates that a compound of interest is a treatment candidate for the treatment of stroke.

2. The method according to claim 1 wherein the compound of interest is a sigma receptor agonist.

3. A method of in vitro screening of compounds for the treatment of stroke comprising the steps of:
inducing ischemia in a population of in vitro neurons;
contacting the neurons with a compound of interest after the induction of ischemia; and
measuring neuronal death at about 6 or more hours after the induction of ischemia, whereby a reduction in neuronal death indicates that a compound of interest is a treatment candidate for the treatment of stroke.

4. The method according to claim 3 wherein ischemia is induced with azide/deoxyglucose.

5. The method according to claim 3 wherein the compound of interest is a sigma receptor agonist.

6. The method according to claim 3 wherein the neurons are contacted with the compound of interest at about 2 or more hours after the induction of ischemia.

7. The method according to claim 3 wherein the neurons are contacted with the compound of interest at about 3 or more hours after the induction of ischemia.

8. The method according to claim 3 wherein the neurons are contacted with the compound of interest at about 6 or more hours after the induction of ischemia.

9. The method according to claim 3 wherein the neuronal death is measured at about 12 or more hours after the induction of ischemia.

10. The method according to claim 3 wherein the neuronal death is measured at about 24 or more hours after the induction of ischemia.

11. A method of in vitro screening of compounds for the treatment of stroke comprising the steps of:
contacting neurons with lipopolysaccharide to induce an inflammatory response;
contacting the neurons with a compound of interest at about 6 hours after the contact of the neurons with lipopolysaccharide; and
measuring the inflammatory response in the cells at about 24 hours after the induction of the inflammatory response, whereby a reduction in the inflammatory response indicates that a compound of interest is a treatment candidate for the treatment of stroke.

12. The method according to claim 11 wherein the step of measuring the inflammatory response comprises measuring TNF-α levels, whereby a reduction in TNF-α levels indicates a reduced inflammatory response.

13. The method according to claim 11 wherein the step of measuring the inflammatory response comprises measuring nitric oxide levels, whereby a reduction in nitric oxide levels indicates a reduced inflammatory response.

14. A method of in vitro screening of compounds for the treatment of stroke comprising the steps of:
inducing an inflammatory response in an in vitro population of neurons;
contacting the neurons with a compound of interest after the induction of the inflammatory response; and
measuring the inflammatory response in the cells at about 6 or more hours after the induction of the inflammatory response, whereby a reduction in the inflammatory response indicates that a compound of interest is a treatment candidate for the treatment of stroke.

15. The method according to claim 14 wherein the step of measuring the inflammatory response comprises measuring TNF-α levels, whereby a reduction in TNF-α levels indicates a reduced inflammatory response.

16. The method according to claim 14 wherein the step of measuring the inflammatory response comprises measuring nitric oxide levels, whereby a reduction in nitric oxide levels indicates a reduced inflammatory response.

17. The method according to claim 14 wherein the neurons are contacted with the compound of interest at about 2 or more hours after the induction of the inflammatory response.

18. The method according to claim 14 wherein the neurons are contacted with the compound of interest at about 3 or more hours after the induction of the inflammatory response.

19. The method according to claim 14 wherein the neurons are contacted with the compound of interest at about 6 or more hours after the induction of the inflammatory response.

20. The method according to claim 14 wherein the inflammatory response in the neurons is measured at about 12 or more hours after the induction of the inflammatory response.

21. The method according to claim 14 wherein the inflammatory response in the neurons is measured at about 24 or more hours after the induction of the inflammatory response.
